(19) United States
(12) Patent Application Publication
(10) Pub. No.: US 2024/0377375 A1
(43) Pub. Date: Nov. 14, 2024

(54) Network based gas sensor system with daisy chain RJ cable based connectivity for scalability, efficiency, flexibility and safety

(71) Applicant: Maarten P.J. Van Laere, Leuven (BE)

(72) Inventor: Maarten P.J. Van Laere, Leuven (BE)

(21) Appl. No.: 18/426,280

(22) Filed: Jan. 29, 2024

Related U.S. Application Data
(60) Provisional application No. 63/441,754, filed on Jan. 27, 2023.

(51) Int. Cl. G01N 33/00

(52) U.S. Cl. CPC G01N 33/0034; G01N 33/0006

(57) ABSTRACT

A new innovative way for connecting multiple gas sensors to a network enabled base unit; this by using a daisy chained connection between the sensors themselves using simple RJ based cabling. The gas sensor(s) connect to each other using a wired connectivity with or without being connected directly or indirectly to a base unit. The gas sensor in this invention is not a single standalone unit but consists of at least two physical different gas sensors that are connected in a sequential or ring daisy chain directly or indirectly to each other using a wired connection.

### BACKGROUND OF THE INVENTION

Until now when multiple wired gas sensors are deployed connected to a base unit, then they are typically connected in a star shaped concept where each gas sensor individually wires back into a base unit or a device connected to a base unit. This has some disadvantages. One main disadvantages is that each gas sensor would require a port on the base unit or a hub to connect to, limiting the number of gas sensors that can be connected to such a base unit. Second disadvantage is that in terms of cabling, each sensor would need to run a cable back to a base unit making deployments more complex and more expensive. There have been some implementations of daisy chained gas sensors using complex cabling like using terminal blocks or I2C connections.

When daisy chained connectivity is used then it is done using cabling other than a RJ cable based connectivity.

The present invention relates generally to a new kind of connectivity for gas sensors using RJ type of cables such as but not limited to RJ45, RJ12 or RJ11 cables. The invention enables multiple gas sensors to connect simply into each other using a wired RJ type connection in a daisy chain type of connection. At some point of the daisy connection the string of daisy chained sensors is then connected using wireless or wired connection directly or indirectly into a base unit. When wired, then the connection to the base unit can be loop (ring) or non-loop based. The base units are IP networked based.

### BRIEF SUMMARY OF THE INVENTION

The invention generally relates to gas sensors that are wired using a RJ45, RJ12 or RJ11 type of cable to each other in a daisy chain type of connection. Typically but not limited to, this enables to have multiple sensors connected to each other in a simple manner. At some point of the daisy connection the string of daisy chained sensors is then connected using a wireless or wired connection directly or indirectly into a base unit.

In electrical and electronic engineering, a daisy chain refers to a wiring scheme in which multiple devices are wired together in sequence or in a ring. The daisy chain in this invention could be a sequence with just one cable starting from the base unit or a ring (also known as a closed loop) whereby the cable returns to the base unit.

There has thus been outlined, rather broadly, some of the features of the invention in order that the detailed description thereof may be better understood, and in order that the present contribution to the art may be better appreciated. There are additional features of the invention that will be described hereinafter.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

Usually when using wired connections, each sensor connects to an available port of a base unit or an expansion unit thereof. As a result of this, the number of gas sensors that can be connected to a base unit is therefore limited to the number of sensor ports available. An object is to provide a new kind of wiring for gas sensors which enables to wire to a base unit on or fewer ports on the base unit, or to another device between the sensor daisy chain wiring string and base unit. This enables the increase of the number of sensors that could otherwise be connected to the physical ports that are available on a base unit.

Another object of this invention is whereby multiple gas sensors are connected to each other on a string without connecting to a base unit. Following is a non-limitative example: a daisy chain connectivity could be useful to share power across all connected sensors and with each sensor having its own outbound IP or non-IP based connectivity. This removes the need in this example for a base unit.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1: FIG. 1 is a drawing of a possible operation of the present invention. Operation of the gas sensors that connect to each other using a wired connection using a RJ45 in a daisy chain type of connectivity. In its turn the sensors then connect to a base unit.

FIG. 2: FIG. 2 is a drawing of a network of sensors and base units including a computer to gather and analyze the data of the sensors and control units.

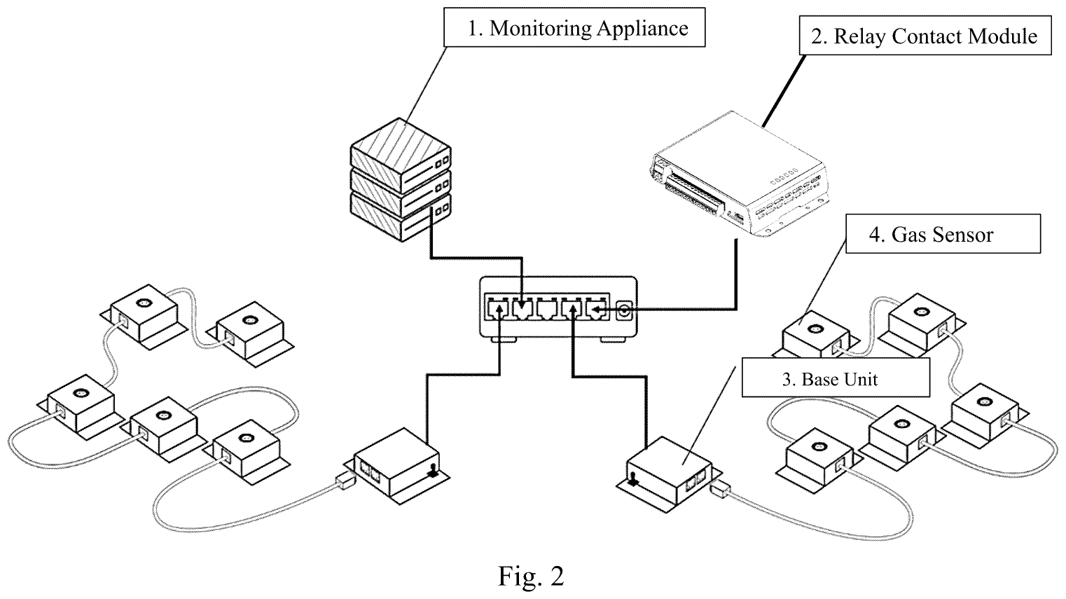

### INDEX OF ELEMENTS

1: Gas Sensor

2: Daisy Chain Connection

3: RJ45 or RJ11 cabling

4: Port

5: Base Unit

### DETAILED DESCRIPTION OF THE INVENTION

### A. Overview

Turning now descriptively to the drawings, in which similar reference characters denote similar elements throughout the several views, the figures illustrate multiple gas sensors that are connected to a base unit in a daisy chained wiring type of connection.

### B. Gas Sensors

The gas sensor consists of a unit that has one or more gas detection components embedded into the unit. A non-limitative example of such a sensor could be a gas sensor capable of detecting the level of H2 or VOC parts present in the ambient air.

### C. Wired Connection

The gas sensor connects to other gas sensors using a sequential wired connection creating a daisy chain of sensors. This sequence wiring can be looped back at the end of the string to form a ring.

In electrical and electronic engineering, a daisy chain is a wiring scheme in which multiple devices are wired together in sequence or in a ring. A ring is whereby a daisy chained wiring loops back to the first device in the chain. A daisy chained connection can be looped or not. The benefit of the loop is that it ensures that if one sensor on the daisy chain fails, that the daisy chain is not broken for the other sensors behind the failing sensor.

The daisy chained sensors can be all gas sensors or could be mixed with sensors of another type in a daisy chain, or one or more other non-sensor type of devices that are connected in the same daisy chain connection string.

The wired connection may transmit data only, power only or may transmit data and power across its RJ cable type to which the different sensors are connected to each other.

### D. Daisy Chain Cabling

The base unit is a device acting as the logical unit and that in turns connects to other systems directly or indirectly.

There are several different types of RJ (Registered Jack) cables, including: RJ11: This is a 4-pin cable that is commonly used for telephone connections; RJ12: This is a 6-pin cable that is similar to RJ11 but with two additional pins; and RJ45: This is an 8-pin cable that is commonly used for Ethernet connections.

RJ cables are a good choice for connecting sensors to transmit power and data because they are widely available and inexpensive. They are also relatively easy to terminate, which makes them suitable for use in a variety of applications.

RJ45 is a good choice for gas sensors in particular because it has an 8-pin connector, which allows for power and data to be transmitted over separate wires. This can be beneficial for ensuring that the sensor is receiving the correct amount of power, and that the data being transmitted is accurate.

Overall, RJ cables are a cost-effective and reliable option for connecting sensors, and the RJ45 cables have the added benefits of being widely available and well-suited for transmitting power and data in gas sensor networks.

### E. Base Unit and Sensor Port

The base unit is a device acting as the logical unit and that in turns connects to other systems directly or indirectly.

The base unit has one or more ports to which one or more sensors can connect to. In other embodiments in this invention, sensors may first connect to one or more devices which in turn are then directly or indirectly connected to a base unit.

In another embodiment, the sensors may use more than one port to connect to. For example, but not limited to this example, this could be required to increase the amount of power sent to the sensors when power on a single port is too limited for the daisy chain to work as intended.

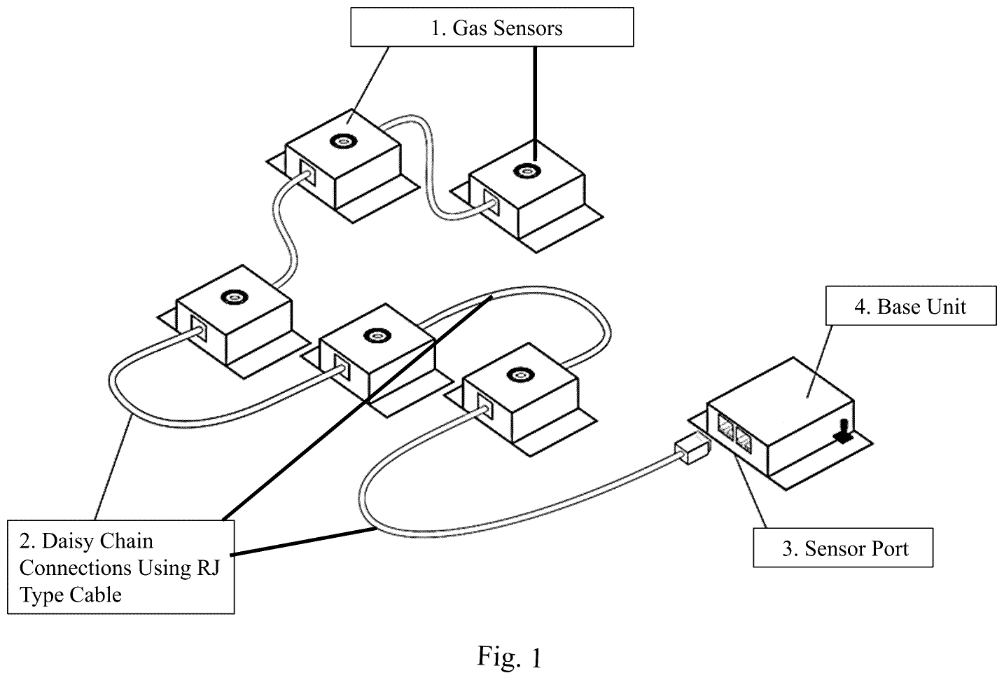

The role of a base unit may be as a separate physical device as per FIG. 1, or the base unit may be combined with the features of a gas sensor within one and the same physical device.

### F. Connections of Main Elements and Sub-Elements of Invention

A typical implementation of this invention would be a sensor device that includes the features as described in the first element. This sensor device would then connect to another sensor device of the same type of sensor or another type, which in turn then connects to a base unit.

On a daisy chain wiring string there could be multiple sensors of the same or different types, as well as other non-sensor type of devices. Such devices could be, but not limited to, devices like signal control and routing devices.

The base (logical) unit connects to the IP network and makes the data available via IP network or serial protocols to applications and other systems.

As previously explained, the base unit could be a separate physical device of the sensor (gas or of any other type) or could be a feature of a sensor (gas or of any other type).

The daisy chained gas sensors could also connect a base unit that is not connected to an IP network.

### G. Operation of Preferred Embodiment

The invention is for a gas sensor that connects to one or more gas sensors using a daisy chained wired connection. In turn it is then connected to one or more base units. In a typical embodiment there would only be one base unit but specific use cases of this invention could require multiple base units on a daisy string connection of gas sensors.

In a traditional way of connecting sensors, one would need to connect each sensor to a port of at least one base unit or any type of device between the sensors and one or more base units. By using a daisy chained wiring connection from one gas sensor to the other, one can have multiple sensors connected to just one sensor port on a base unit or port on a device in between the sensors and a base unit like, but not limited to, a sensor hub.

In another embodiment of the invention, the gas sensors could be all directly or indirectly connected to each other without connecting to a base unit. For example, the wiring cable connecting all units together in sequence would transmit power across all connected sensors and devices with each having its own IP or non-IP based connectivity to other systems.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention in which all terms are meant in their broadest, reasonable sense unless otherwise indicated. Any headings utilized within the description are for convenience only and have no legal or limiting effect.

To update and improve the patent application with the additional information provided, we need to integrate the new elements into the existing structure of the document, ensuring clarity and completeness. Here's how the modified sections and additional claims might look:

### H. Integration with Other Sensor Types

In addition to gas sensors, this system can incorporate other types of sensors within the same daisy chain or in a different daisy chain connected to the same or different base unit. This includes, but is not limited to, thermal, environmental, power, or mechanical sensors. This integration allows for a more comprehensive monitoring and anomaly detection system.

### I. Data Analysis and Decision Making

The system can interface with one or more advanced data analysis units, such as the ServersCheck Monitoring Appliance. This computer analyzes the data collected from all sensors connected to various base units. It employs various decision-making frameworks, including rule-based systems, predictive learning, machine learning, artificial intelligence, or any combination thereof. This enables sophisticated data interpretation and actionable insights. It could be on-premise or could be cloud based or a combination thereof for all or part of the functionality.

### J. Resource Sharing Across Network

Networked base units in this system can share resources. For example, a relay or contact device connected to one base unit can be activated by commands from other base units. This extends to other IP-enabled devices or those connected to the central data analysis computer, facilitating coordinated actions across the network. Such a module could also be connected to the network and accessed by either the computer or the base units. As it is an IP based network this could be more than just one device that have similar or different functionality.

1. A network-based sensor system comprising multiple base units with directly or indirectly connected sensors, including gas sensors and sensors of other types such as thermal, environmental, power, or mechanical sensors, connected in a daisy chain configuration using RJ-type cables.

2. The system of claim 1, wherein the sensors in the daisy chain configuration are capable of transmitting both data and power through the RJ-type cables.

3. The system of claim 1, further including a data analysis unit capable of processing data from the sensors using decision-making frameworks that include rule-based systems, predictive learning, machine learning, artificial intelligence, or combinations thereof.

4. The system of claim 1, wherein the base units are capable of sharing resources across the network, allowing for coordinated control and interaction with devices connected to different base units.

5. The system of claim 4, wherein the shared resources include, but are not limited to, relays, contact devices, and IP-enabled devices.

6. The system of claim 1, where the base units are capable of interfacing with an IP network to enable data sharing, remote management, and coordination of actions across the network.

7. A method of monitoring and detecting anomalies using the system of any preceding claims, wherein data from various sensor types is collected, analyzed, and used to trigger responses based on predefined criteria or learned patterns either by one or more base units or one or more dedicated computer system or any other logical unit.

8. The system of claim 7, wherein the daisy chain configuration includes a loop-back at the end of the sensor string to form a ring, ensuring continuity of operation in case of a sensor failure.

9. The system of claim 7, wherein the daisy chain configuration includes non-sensor devices such as signal control and routing devices, enhancing the functionality and flexibility of the network.

10. The system of claim 7, wherein the configuration allows for dynamic reconfiguration of the sensor network in response to detected anomalies or environmental changes.

11. The system of claim 7, wherein the sensors and base units are configured to automatically update their firmware and software, ensuring ongoing compatibility and performance optimization.

12. The system of claim 7, where the base units and sensors are equipped with self-diagnostic capabilities, enabling proactive maintenance and troubleshooting.

13. The system of claim 12, wherein the self-diagnostic capabilities include predictive maintenance alerts based on sensor performance data and historical trends.
